(19) United States
(12) Patent Application Publication
(10) Pub. No.: US 2011/0201048 A1
(43) Pub. Date: Aug. 18, 2011

(54) SCREENING METHOD OF THERAPEUTIC AND DIAGNOSTIC AGENTS FOR TNF-ALPHA-INDUCED DISEASES USING REACTIVE OXYGEN SPECIES MODULATOR 1

(75) Inventors: Young-Do Yoo, Seoul (KR); Jung-Jin Kim, Seoul (KR)

(73) Assignee: KOREA UNIVERSITY RESEARCH AND BUSINESS FOUNDATION, Seoul (KR)

(21) Appl. No.: 13/126,421

(22) PCT Filed: Jan. 11, 2010
(86) PCT No.: PCT/KR10/00141

§ 371 Date: Apr. 27, 2011

(30) Foreign Application Priority Data
Jul. 8, 2009 (KR) 10-2009-0062255

(51) Int. Cl. C12Q 1/02; G01N 33/50; G01N 33/53

(52) U.S. Cl. 435/29; 436/94; 436/501

(57) ABSTRACT

Disclosed are methods for screening therapeutics, prophylactics or pain alleviators for diseases induced by TNF-α, a main mediator of inflammation and in ROS production and cell death, and for diagnosing the diseases, and a diagnostic kit. For this, a nucleotide sequence and an amino acid sequence of Romo1, which plays an important role in the ROS production and cell death pathway of TNF-α signaling are utilized.

### TECHNICAL FIELD

The present invention relates to a method of screening a therapeutic, a prophylactic or a pain alleviator for TNF-α-induced diseases, and a diagnostic method and a diagnostic kit for TNF-α-induced diseases.

### BACKGROUND ART

Inflammation is the primary biological response of a tissue to harmful stimuli, such as pathogens, damaged cells, or irritants. Inflammation is a protective attempt by the organism to remove the injurious stimuli as well as to initiate the tissue's healing process. TNF-α, interleukin-1 and interleukin-6 are known as the main factors responsible for inducing inflammation.

TNF-α is a major mediator of inflammation and plays an important role in tissue regeneration/expansion and destruction during inflammation (Wajant H. et al. Cell Death and Differentiation 10: 45-65, 2003). In a normal state, inflammation is well regulated by these factors. That is, after these factors cause inflammation with the concomitant induction of immune responses, their levels decrease to a normal state.

However, deregulated TNF-α production causes chronic inflammation, which is directly associated with a variety of diseases such as arthritis, sclerosis, Alzheimer's disease, fibrosis, cancer, diabetes, inflammatory bowel diseases, etc. (Balkwill F. et al. Nature 431:405-406, 2004; Chen G and David V. G. Science 296: 1634, 2002).

Below, a description is given of the induction of chronic inflammation by TNF-α.

There are two main pathways in TNF-α signaling: cell survival pathway (NF-kβ pathway) and cell death pathway (ROS-JNK-caspase pathway) (Papa S. et al. Cell Death and Differentiation 13: 712-729, 2006). During TNF-α signaling, reactive oxygen species (hereinafter referred to as “ROS”) are generated. The main source of ROS generation is the mitochondria, but the exact mechanism of mitochondrial ROS still remains unknown (Chandel N. S. et al. J. Biol. Chem. 276: 42728-42736, 2001; Hennet T. et al. Cancer Res. 53: 1456-1460, 1993; Hennet T. et al. Biochem. J. 289: 587-592, 1993; Schulze-Osthoff K. et al. J. Biol. Chem. 267: 5317-5323, 1992).

In organism, ROS of proper amount plays an important role in signaling pathways. However, when increasing in intracellular level due to external stress, ROS causes a variety of diseases including cancer, aging, inflammation, diabetes, arteriosclerosis, hepatic fibrosis, etc. (Droge W., Physiol. Rev. 82: 47-95, 2002). Thus, ROS is reported to arise as a molecular target to treat inflammatory diseases. ROS is known to play an important role in inflammatory pain as well as acute and chronic inflammation (Salvemini D. et al. Biochem. Soc. Trans. 34: 965-970, 2006).

ROS generated in TNF-α signal pathway is involved in NF-kB activation and TNF-α-induced cell death. Particularly, excessive ROS generated by deregulated TNF-αproduction is closely correlated with TNF-α-induced chronic inflammation (Sulciner D. J. et al. Mol. Cell. Biol. 16: 7115-7121, 1996; Schulze-Osthoff K. et al. EMBO J. 12: 3095-3104, 1993) (Leist et al. Nat. Rev. Mol. Cell. Biol. 2: 589-598, 2001).

An animal experiment demonstrated that TNF-α is a major mediator of inflammation. Transgenic mice with a TNF-α gene inserted thereto suffered from arthritis (Keffer J. et al. EMBO J. 10: 4025-4031, 1991). So far, therefore, the blocking of TNF-α function has been reported to be the most effective therapy for arthritis (Olsen N. J. and Stein C. M. N Engl J. Med. 350: 2167-2179, 2004).

Among the drugs, developed thus far, for targeting TNF-α are Infliximab (a chimeric monoclonal antibody against human TNF), Adalimumab (a fully human monoclonal antibody), Etanercept (a dimeric TNFRII (p75) fusion protein linked to the Fc portion of human IgG), Golimumab, CDP571, and Thalidomide (Bongartz T. et al. JAMA. 295: 2275-2482, 2006). Also inhibiting the positive functions of TNF-α, these drugs may elicit unwanted outcomes (Andrei A. et al. Cytokine & Growth Factor Reviews 19: 231-244, 2008) including lymphoma development and infection (Faubion W. A. et al. Clin Gastroenterol Hepatol 4: 1199-1213, 2006).

There is therefore a need for an agent that regulates the excessive ROS generation and cell death which is induced by TNF-α without blocking the positive physiological functions of TNF-α.

### DISCLOSURE OF INVENTION

### Technical Problem

Accordingly, the present invention has been made keeping in mind the above problems occurring in the prior art, and an object of the present invention is to provide a method of screening a therapeutic, a prophylactic or an analgesic for TNF-α-induced inflammatory diseases, which can suppress TNF-α-mediated ROS generation and cell death without inhibiting the positive physiological functions of TNF-α. The medicinal system provided by the present invention targets a ROS generation protein derived from the Romo1 gene and protein, which plays an important role in an intermediate signaling pathway when TNF-α induces inflammatory diseases. Therefore, the medicinal system of the present invention is the system for screening agents capable of regulating the intermediate signaling pathway and for regulating Romo1 which is the origin of mitochondrial ROS production, not removing the excessive ROS already generated by Romo1.

It is another object of the present invention to provide a method and a kit for diagnosis of TNF-α-induced diseases.

### Solution to Problem

In accordance with an aspect thereof, the present invention provides a method for screening a therapeutic, a prophylactic or a pain alleviator for TNF-α-induced diseases, comprising selecting an agent specific for a gene having the nucleotide sequence of SEQ ID NO. 1 (hereinafter referred to as “Romo1 gene”) or an analog thereof:

*SEQ ID NO. 1:
GAGCGCCCTCCCCGTCGTTTTCCGTGAGAGACGTAGAGCTGAGCGACCCA
GCCCGCGAGCGAGGTGAGATGCCGGTGGCCGTGGGTCCCTACGGACAGTC
CCAGCCAAGCTGCTTCGACCGTGTCAAAATGGGCTTCGTGATGGGTTGCG
CCGTGGGCATGGCGGCCGGGGCGCTCTTCGGCACCTTTTCCTGTCTCAGG
ATCGGAATGCGGGGTCGAGAGCTGATGGGCGGCATTGGGAAAACCATGAT
GCAGAGTGGCGGCACCTTTGGCACATTCATGGCCATTGGGATGGGCATCC
GATGCTAACCATGGTTGCCAACTACATCTGTCCCTTCCCATCAATCCCAG
CCCATGTACTAATAAAAGAAAGTCTTTGAGTAAAAAAAAAA

In accordance with another aspect thereof, the present invention provides method for screening a therapeutic, a prophylactic or a pain alleviator for TNF-α-induced diseases, comprising selecting an agent specific for a protein, encoded by the Romo1 gene, having the amino acid sequence of SEQ ID NO. 2 or an analog thereof.

*SEQ ID. NO. 2:
MPVAVGPYGQSQPSCFDRVKMGFVMGCAVGMAAGALFGTFSCLRIGMRGR
ELMGGIGKTMMQSGGTFGTFMAIGMGIRC

In accordance with a further aspect thereof, the present invention provides a method for diagnosing TNF-α-induced diseases, comprising determining an mRNA expression level of the Romo1 gene.

In accordance with still a further aspect thereof, the present invention provides a method for diagnosing TNF-α-induced diseases, comprising determining a protein expression level of the Romo1 gene.

In accordance with still another aspect thereof, the present invention provides a diagnostic kit of TNF-α-induced diseases, utilizing the diagnosing method.

### Advantageous Effects of Invention

Featuring specificity for a Romo1 gene or derivatives thereof or a Romo1 protein or derivatives thereof, the screening methods of therapeutics, prophylactics or pain alleviators for TNF-α-induced diseases in accordance with the present invention can be used to develop anti-inflammatory agents which target TNF-α and show high medicinal efficacy without side effects. In addition, Romo1 genes or proteins may serve as useful materials in kits, DNA chips and proteins chips for use in diagnosing TNF-α-induced diseases. Accordingly, the methods of the present invention are not directed to the removal of already produced ROS, but pertain to the blocking or regulation of the origin of ROS production responsible for diseases.

Based on the present invention, therefore, a therapeutic, a prophylactic or a pain alleviator for TNF-α-induced diseases including sepsis, diabetes, osteoporosis, allograft rejection, multiple sclerosis, rheumatoid arthritis, inflammatory bowel diseases, ischemia/reperfusion injury, pulmonary fibrosis, and neurodegenerative disorders (ischemic stroke, Alzheimer's disease, Parkinson's disease), which can suppress the ROS production and cell death induced by TNF-α without blocking the positive physiological functions of TNF-α, can be screened. Also, diagnostic methods and kits may be provided.

### BRIEF DESCRIPTION OF DRAWINGS

FIG. 1 is a photograph taken after HEK 293 cells treated with TNF-α were subjected to Western blotting, showing that in response to TNF-α, Romo1 is associated with the complex II members RIP, TRADD, TRAF2 and FADD, which are involved in the cell death pathway of TNF-α signaling pathway.

FIG. 2 is a photograph taken after HEK 293 cells treated with TNF-α were subjected to Western blotting, showing that in response to TNF-α, Romo1 is associated with Bcl-XL, which functions to stabilize the mitochondrial membrane potential.

FIG. 3 is a graph showing that Romo1 reduces the mitochondrial membrane potential in response to TNF-α signals in HeLa cells. Romo1 knockdown by Romo1 siRNA (100 nM) blocks the TNF-α-induced reduction of the mitochondrial membrane potential.

### BEST MODE FOR CARRYING OUT THE INVENTION

In accordance with an aspect thereof, the present invention is directed to a screening method of therapeutics, prophylactics or pain alleviators for TNF-α-induced diseases, comprising selecting agents specific for an Romo1 gene.

In accordance with another aspect thereof, the present invention is directed to a screening method of therapeutics, prophylactics or pain alleviators for TNF-α-induced diseases, comprising selecting agents specific for a protein having the amino acid sequence of SEQ ID NO. 2 (i.e., Romo1) or an analog thereof.

As used herein, the term “analog” of the nucleotide sequence of SEQ ID NO. 1 means a gene which partially differs in nucleotide sequence from the Romo1 gene as a result of deletion, substitution or addition, but encodes a protein functionally identical or similar to Romo1 functioning as a mediator of the TNF-α-mediated cell death pathway.

As used herein, the term “a protein having the amino acid sequence encoded by the Romo1 gene” means a protein having the amino acid sequence of SEQ ID NO. 2, which is encoded by the open reading frame of the cDNA of SEQ ID NO. 1 to the exclusion of 5′- and 3′-UTR, corresponding to the nucleotide stretch of from n.t. 69 to n.t. 305 (i.e., underlined in SEQ ID NO. 1).

As used herein, the term “TNF-α-induced diseases” is intended to refer to diseases resulting from the deregulated ROS production induced by TNF-α binding. Among them are sepsis, diabetes, osteoporosis, allograft rejection, multiple sclerosis, rheumatoid arthritis, inflammatory bowel diseases, ischemia/reperfusion injury, pulmonary fibrosis, and neurodegenerative disorders (ischemic stroke, Alzheimer's disease, Parkinson's disease).

TNF-α is a major factor of inflammation with ROS serving as a mediator. Because they directly target TNF-α, conventional therapeutics for TNF-α-induced diseases inhibit the positive physiological functions of TNF-α, causing various side effects.

The research lab of the present inventors was the first in the world to discover a novel protein which functions as a mediator of the TNF-α-induced cell death pathway and named it Romo1 (reactive oxygen species modulator 1). Increasing in intracellular level in response to external stress, Romo1 was found to stimulate TNF-α-induced ROS production and apoptotic cell death. In full consideration of this finding, experiments demonstrated that the regulation of Romo1 level suppressed TNF-α-induced ROS production and apoptotic cell death. Based on the experimental data, an agent or drug capable of regulating Romo1 activity was found to be also effective in blocking TNF-α-mediated inflammation.

In other words, the present invention is not directed to the removal of already produced ROS, but pertains to the blocking or regulation of the origin of ROS production responsible for diseases. In this regard, the screening system developed according to the present invention targets agents or factors specific for Romo1, but not ROS scavengers.

The screening method according to the present invention comprises the following two steps:

1) treating cells with a test compound of interest, together with TNF-α in such an amount as to induce cell death while treating separate cells of the same amounts with a control antioxidant, together with TNF-α in the same amount; and

2) selecting the test compound as an effector if it shows an antioxidant activity similar to that of the control with concomitant blocking of decrease of mitochondrial membrane potential compared to an initial potential.

The cells useful in the screening method of the present invention may be from mammals, such as humans, pigs, cows, rabbits, mice, etc.

The antioxidant activity may be determined by measuring ROS levels in the cells. Giving that the ROS levels in the test group treated with a test compound and TNF-αand in the control treated with an antioxidant and TNF-α are a and b, respectively, a similar antioxidant effect may be determined in step 2) when the two values are comparable, preferably when a and b meet the following formula: a×0.8≦b≦a×1.2 (i.e., b is within a±20%).

On the other hand, the term “mitochondrial membrane potential” means the potential of the mitochondrial membrane in which Romo1 is located. Giving that the initial mitochondrial membrane potential and the mitochondrial membrane potential of the experimental group are c and d, respectively, the mitochondrial membrane potential is determined to block decrease of potential in step 2) when the two potentials meet the following formula: c≦d

Particularly when targeting an agent specific for Romo1 protein, but not for Romo1 gene, the screening method of the present invention comprises:

1) treating cells with a test compound of interest, together with TNF-α in such an amount as to induce cell death while treating separate cells of the same amounts with a control antioxidant, together with TNF-α in the same amount; and

2) selecting the test compound as an effector if it suppresses the Romo1 protein from binding to a complex II member selected from the group consisting of RIP1, TRAF2, TRADD, FADD and a combination thereof or from associating with Bcl-xL in higher efficiency compared to the control.

This screening method is based on the following findings.

There are two main pathways in TNF-α signaling: a cell survival pathway and a cell death pathway. In the cell death pathway, ROS is produced from mitochondria while the expression of FHC and Mn-SOD is induced to eliminate ROS in the cell survival pathway (Papa S. et al. Cell Death and Differentiation 13: 712-729, 2006).

The treatment of cells with the translation inhibitor cycloheximide (CHX) suppresses the expression of FHC and MnSOD, resulting in accumulating the ROS produced by TNF-α with time (control siRNA in Tables 1 and 2).

After treatment with CHX, when the cells were decreased in Romo1 expression level by use of Romo1 siRNA, the ROS production induced by TNF-α was observed to cease (Romo1 siRNA in Tables 1 and 2). In this experiment, the antioxidant BHA was used as a positive control (BHA in Tables 1 and 2).

Taken together, the experimental data indicate that Romo1 is a major mediator of TNF-α-induced ROS production. TNF-α-triggered cell death is directly associated with TNF-α-induced inflammatory diseases (Leist M and Jaattela M. Nat. Rev. Mol. Cell. Biol. 2: 589-598, 2001), examples of which include sepsis, diabetes, osteoporosis, allograft rejection, multiple sclerosis, rheumatoid arthritis, inflammatory bowel diseases, ischemia/reperfusion injury, pulmonary fibrosis, and neurodegenerative disorders.

Romo1 is involved in TNF-α-triggered cell death pathway. To demonstrate this, cells were co-treated with CHX and TNF-α in the presence or absence of Romo1 siRNA. 9 Hours after co-treatment with CHX and TNF-α alone, the cells died in about 89% of the population. In contrast, the cells which were transfected with Romo1 siRNA to decrease Romo1 expression levels experienced TNF-α-triggered cell death only in about 50% of their population (Romo1 siRNA in Table 3). On the other hand, TNF-α-triggered cell death was observed only in about 48% of the cell population in the presence of BHA (BHA in Table 3).

These data suggest that agents inhibitory of Romo1 can be used as anti-inflammatory agents useful in the treatment of TNF-α-induced inflammation.

Because Romo1 is involved in ROS production, a screening system for agents for ROS elimination, such as that for measuring cell death rate (i.e., for determining antioxidant activity), is not discriminated from conventional screening systems of antioxidants, and thus does not feature Romo1.

In order to overcome this problem, an examination was made of the mechanism by which TNF-α produces ROS the mitochondria via Romo1.

In response to TNF-α, complex II (consisting of RIP1, TRADD, TRAF2, FADD, and pro-caspase-8) is formed to induce cell death.

In the present invention, complex II was found to bind to Romo1 located in the mitochondria to mediate TNF-α signaling.

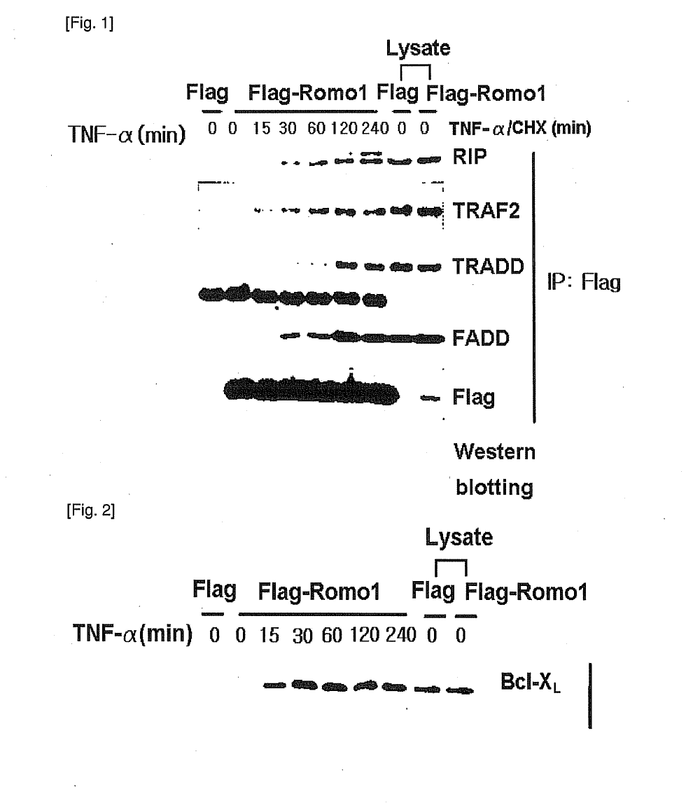

Upon treatment with TNF-α, the interaction of Romo1 with RIP, TRADD, TRAF2 and FADD were observed to increase (FIG. 1). Concurrently, Romo1 recruits Bcl-XL, known to stabilize mitochondrial membrane potential and to suppress cell death, to increase ROS level and reduce mitochondrial membrane potential. That is, Bcl-XL was functionally interrupted, resulting in a decrease in mitochondrial membrane potential from 0.84 to 0.59 (control siRNA in FIG. 3). The initial ROS thus produced, if not eliminated by FHC or MnSOD as a result of treatment with CHX, further decreases the mitochondrial membrane potential, further giving rise to an increase in ROS production.

As is apparent from data of FIG. 3, Romo1 knockdown by Romo1 siRNA blocked the TNF-α-induced decrease of mitochondrial membrane potential 15 min after TNF-αtreatment (from 1.34 to 1.44) while the antioxidant BHA could not prevent the decrease of mitochondrial membrane potential (from 1.17 to 0.74).

Accordingly, the system of screening an agent which can show an antioxidation activity while keeping the mitochondrial membrane potential high can be used as a specific screening system of Romo1 inhibitors, which is quite different from conventional antioxidant-screening systems.

Additionally, the screening system of the present invention includes the system of screening agents which block the interaction of Romo1 with a member of complex II, e.g., RIP1, TRAF2, TRADD or FADD, or with Bcl-XL.

In accordance with a further aspect thereof, the present invention is directed to a diagnostic method of TNF-α-induced diseases, comprising determining the level of Romo1 mRNA.

In accordance with still a further aspect thereof, the present invention is directed to a diagnostic method of TNF-α-induced diseases, comprising determining the expression level of Romo1 protein.

As described above, it should be understood that those of ordinary skill in the art can readily apply the screening methods of therapeutics, prophylactics or pain alleviators for TNF-α-induced diseases to the diagnostic methods.

For example, the quantification of Romo1 mRNA level may lead to the diagnosis of TNF-α-induced diseases.

The Romo1 mRNA level can be determined by quantitatively measuring the oligonucleotide which hybridizes at least partially with the Romo1 gene.

Also, the quantification of Romo1 protein level can lead to the same result.

Based on the diagnostic method, a diagnostic kit is provided in accordance with the present invention.

### MODE FOR THE INVENTION

A better understanding of the present invention may be obtained through the following examples which are set forth to illustrate, but are not to be construed as limiting the present invention.

### EXAMPLES

### Example 1

### Cell Culture

HeLa and HEK 293 cells were purchased from the American Type Culture Collection (ATCC) and cultured in 10% DMEM/F12 culture media (GIBCO/BRL, Grand Island, N.Y.), supplemented with 10% fetal bovine serum, containing 100 units/ml of penicillin and 100 μg/ml of streptomycin.

### Example 2

### Romo1 siRNA Transfection

Cells were seeded at a density of 1×10⁵ cells/well into 6-well plates. After incubation for 24 hrs, the cells were washed once with an antibiotic-free, serum-free medium and transfected with control siRNA or Romo1 siRNA (100 nM) using Lipofectamine (5 μL). 6 Hours after the transfection, the cells were washed twice with a serum-free medium and cultured in serum media.

Romo1 siRNA had the following nucleotide sequence: 5′-CUA CGG ACA GUC CCA GCC A (dTdT)-3′ (sense) and 5′-UGG CUG GGA CUG UCC GUA G (dTdT)-3′(antisense).

### Example 3

### ROS Assay

Cover glasses coated with 0.1% gelatin were placed on 6-well plates in which cells were then seeded at a density of 2×10⁴ cells/well. After incubation for 24 hrs, the cells were treated with CHX and TNF-α. For quantitative assay of ROS, the cells were treated for 30 min with 10 μM of the ROS probe DCF-DA (2,7-dichlorofluorescein diacetate) in a light-tight condition, incubated in a 37° C. incubator, and washed twice with PBS. On the slide glass was dropped 20 μL of a mounting medium (H-1000, Vector shield, Vector Laboratories, CA) which was then covered with the cell-fixed cover glass. The cover glass was mounted on the slide glass using transparent manicure and dried before observation under a fluorescence microscope (Olympus LX71 microscope). ROS was quantitatively determined using Metamorph software (Universal Imaging, Westchester, Pa.).

### Example 4

### Assay for Cell Death Rate Post-TNF-α Treatment

Cells were seeded at a density of 1×10⁵ cells/well into 6-well plates. After incubation for 24 hrs, the cells were washed once with an antibiotic-free, serum-free medium and transfected with control siRNA or Romo1 siRNA (100 nM) using Lipofectamine (5 μL). 6 Hours after the transfection, the cells were washed twice with a serum-free medium and cultured for 24 hours in serum media. Then, the cells were detached from the bottom of the plates by treatment with trypsin-EDTA and suspended at a density of 2×10⁴ cells in 2 mL of a medium in each well of 6-well plates. After treatment with TNF-α and CHX, cell viability was determined by trypan blue (0.4%) staining.

### Example 5

### Immunoprecipitation Assay and Immunoblotting

Cells were transfected with Flag-tagged Romo1, followed by lysis in RIPA buffer (NaCl 150 mM, 1% Nonidet P-40, 0.5% deoxycholic acid, 0.1% SDS, Tris 50 mM, pH 8.0) containing phosphate buffer (1 mM sodium orthovanadate, NaF 30 mM, phenyl methylsulfonyl fluoride 1 mM, NaPPi 30 mM). The cell lysates prepared in the RIPA buffer were measured for protein concentration using a protein assay kit (BioRad, Hercules, Calif.). Incubation with an anti-FLAG antibody formed an antibody-Flag-tagged Romo1, followed by incubation with protein A agarose to form a complex of Romo1-Frag/antibody/protain A agarose. This complex was spun down (4° C., 1 min, 5,000 rpm) and washed three times with PBS (phosphate-buffered saline), followed by separation on SDS-polyacrylamide gel using electrophoresis. After being transferred onto nitrocellulose membranes, the immunoprecipitants were subjected to Western blotting against respective antibodies to RIP, TRADD, TRAF2, FADD, Bcl-xL, and Flag.

### Example 6

### Measurement of Mitochondrial Membrane Potential

Cells were seeded at a density of 1×10⁵ cells/well into 6-well plates. After incubation for 24 hrs, the cells were washed once with an antibiotic-free, serum-free medium and transfected with control siRNA or Romo1 siRNA (100 nM) using Lipofectamine (5 μL). 6 Hours after the transfection, the cells were washed twice with a serum-free medium and cultured in serum media. To determine changes in mitochondrial membrane potential, the cells treated with TNF-α (20 ng/ml) in the presence or absence of CHX were incubated for 15 min with JC-1 (5 μM) and washed twice with PBS, immediately followed by flow cytometry. Images were collected on the FL1 channel for green JC-1 monomers and on the FL2 channel for red JC-1 aggregates. Changes in mitochondrial membrane potential were determined using red/green ratios

Data obtained in Examples 1 to 6 are discussed, below.

Table 1 shows that when the Romo1 expression level is reduced with Romo1 siRNA, the ROS production induced by TNF-α (20 ng/ml) is blocked in HeLa cells. In this regard, the cells treated with TNF-α were observed under a fluorescent microscope and quantitatively measured for ROS. In Table 1, the ROS amounts produced with time after TNF-α treatment were expressed as ratios to the ROS amount produced without TNF-α. The antioxidant BHA (butylated hydroxyanisole) was used as a positive control.

Control siRNA: relative ROS amounts upon treatment with control siRNA (100 nM).

Romo1 siRNA: relative ROS amounts upon treatment Romo1 siRNA (100 nM).

BHA: relative ROS amounts upon treatment with BHA (100 μM) 30 min before TNF-α treatment.

The cells were incubated with Cycloheximide (CHX) (10 μg/ml) 30 min before TNF-α treatment.

TABLE 1

| | | | |
|---|---|---|---|
| Time | Control siRNA | Romo1 siRNA | BHA |

| 0 | | 1.0 | 1.0 | 1.0 |
|---|---|---|---|---|
| 15 | min | 1.0 | 1.1 | 1.1 |
| 30 | min | 1.2 | 1.3 | 1.1 |
| 1 | hr | 1.4 | 1.3 | 1.1 |
| 2 | hrs | 1.5 | 1.1 | 1.2 |
| 4 | hrs | 2.1 | 1.2 | 1.0 |
| 6 | hrs | 2.5 | 1.3 | 1.0 |

Table 2 shows that Romo1 knockdown by Romo1 siRNA blocks the ROS production induced by TNF-α (20 ng/ml) in HEK 293 cells. In this regard, the cells treated with TNF-α were observed under a fluorescent microscope and quantitatively measured for ROS. In Table 2, the ROS amounts produced with time after TNF-α treatment were expressed as ratios to the ROS amount produced without TNF-α. The antioxidant BHA (butylated hydroxyanisole) was used as a positive control.

Control siRNA: relative ROS amounts upon treatment with control siRNA (100 nM).

Romo1 siRNA: relative ROS amounts upon treatment with Romo1 siRNA (100 nM).

BHA: relative ROS amounts upon treatment with BHA (100 μM) 30 min before TNF-α treatment.

The cells were incubated with Cycloheximide (CHX) (10 μg/ml) 30 min before TNF-α treatment.

TABLE 2

| | | | |
|---|---|---|---|
| Time | Control siRNA | Romo1 siRNA | BHA |

| 0 | | 1.0 | 1.0 | 1.0 |
|---|---|---|---|---|
| 15 | min | 1.2 | 1.2 | 1.3 |
| 30 | in | 1.3 | 1.3 | 1.4 |
| 1 | hr | 1.5 | 1.1 | 1.1 |
| 2 | hrs | 2.1 | 1.2 | 1.2 |
| 4 | hrs | 2.4 | 1.2 | 1.1 |
| 6 | hrs | 2.8 | 1.2 | 0.8 |

Table 3 summarizes the results of a cell counting experiment showing that Romo1 knockdown by Romo1 siRNA blocks TNF-α-induced cell death. In Table 3, the ROS amounts produced with time after TNF-α treatment were expressed as ratios to the ROS amount produced without TNF-α. The antioxidant BHA (butylated hydroxyanisole) was used as a positive control.

Control siRNA: relative ROS amounts upon treatment with control siRNA (100 nM).

Romo1 siRNA: relative ROS amounts upon treatment with Romo1 siRNA (100 nM).

BHA: relative ROS amounts upon treatment with BHA (100 μM) 30 min before TNF-α treatment.

The cells were incubated with Cycloheximide (CHX) (10 μg/ml) 30 min before TNF-α treatment.

TABLE 3

| Time | Control siRNA | Romo1 siRNA | BHA |
|---|---|---|---|
| 0 hrs | 1.00 | 1.00 | 1.00 |
| 3 hrs | 0.65 | 0.92 | 0.90 |
| 6 hrs | 0.29 | 0.63 | 0.68 |
| 9 hrs | 0.11 | 0.50 | 0.52 |

FIG. 1 shows the results of Western blotting with HEK 293 cells treated with TNF-α, showing that Romo1 is associated with the complex II members RIP, TRADD, TRAF2 and FADD, which are involved in the cell death pathway of TNF-α signaling. After being transfected with Flag-tagged Romo1, the cells were incubated with anti-Flag antibodies to obtain Romo1-Flag immunoprecipitants. In order to identify proteins that bind to Romo1 with time in response to TNF-α signal, Western blotting was conducted against respective antibodies to RIP, TRADD, TRAF2 and FADD. For Lysate, the cell lysate was subjected to Western blotting without precipitation. The test proteins were used in the same amounts as measured for β-actin.

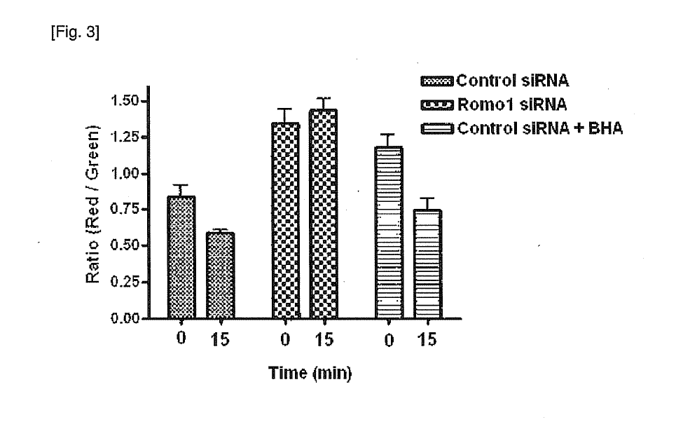

FIG. 2 shows the results of Western blotting with HEK 293 cells treated with TNF-α, showing that Romo1 is associated with Bcl-XL, which functions to stabilize the mitochondrial membrane potential. After being transfected with Flag-tagged Romo1, the cells were incubated with anti-Flag antibodies to obtain Romo1-Flag immunoprecipitants. In order to identify the interaction of Romo1 with Bcl-XL with the passage of time in response to a TNF-α signal, Western blotting was conducted against an anti-Bcl-XL antibody. For Lysate, the cell lysate was subjected to Western blotting without precipitation. The test proteins were used in the same amounts as measured for β-actin.

FIG. 3 shows that Romo1 reduces the mitochondrial membrane potential in response to TNF-α signals in HeLa cells. The cells were incubated for 15 min with 5 μM of the mitochondrial membrane potential probe JC-1 (5,5,6,6-tetrachloro-1,1,3,3-tetraethylbenzimidazolcarbocyanine iodide) and washed twice with PBS (phosphate-buffered saline), immediately followed by flow cytometry. Romo1 knockdown by Romo1 siRNA (100 nM) blocked the TNF-α-induced reduction of the mitochondrial membrane potential whereas the antioxidant BHA did not. Images were collected on the FL1 channel for green JC-1 monomers and on the FL2 channel for red JC-1 aggregates. Changes in mitochondrial membrane potential were determined using red/green ratios. A decrease in red/green ratio means a reduction in mitochondrial membrane potential.

Although the preferred embodiment(s) of the present invention have (has) been disclosed for illustrative purposes, those skilled in the art will appreciate that various modifications, additions and substitutions are possible, without departing from the scope and spirit of the invention as disclosed in the accompanying claims.

1. A method of screening a therapeutic, a prophylactic or a pain alleviator for a TNF-α-induced disease, comprising selecting an agent specific for a DNA having a nucleotide sequence of SEQ ID NO. 1 or an analog thereof.

2. The method according to claim 1, wherein the TNF-α-induced disease is selected from the group consisting of sepsis, diabetes, osteoporosis, allograft rejection, multiple sclerosis, rheumatoid arthritis, inflammatory bowel diseases, ischemia/reperfusion injury, pulmonary fibrosis, ischemic stroke, Alzheimer's disease, Parkinson's disease and a combination thereof.

3. A method of screening a therapeutic, a prophylactic or a pain alleviator for a TNF-α-induced disease, comprising selecting an agent specific for a protein having an amino acid sequence of SEQ ID NO. 2 or an analog thereof.

4. The method according to claim 3, wherein the TNF-α-induced disease is selected from the group consisting of sepsis, diabetes, osteoporosis, allograft rejection, multiple sclerosis, rheumatoid arthritis, inflammatory bowel diseases, ischemia/reperfusion injury, pulmonary fibrosis, ischemic stroke, Alzheimer's disease, Parkinson's disease and a combination thereof.

5. The method according to claim 1 or 3, comprising:
1) treating cells with a test compound of interest, together with TNF-αin such an amount as to induce cell death while treating separate cells of the same amounts with a control antioxidant, together with TNF-α in the same amount; and
2) selecting the test compound as an effector if it shows an antioxidant activity with concomitant blocking of decrease of mitochondrial membrane potential compared to an initial potential.

6. The method according to claim 3, comprising:
1) treating cells with a test compound of interest, together with TNF-αin such an amount as to induce cell death while treating separate cells of the same amounts with a control antioxidant, together with TNF-α in the same amount; and
2) selecting the test compound as an effector if it suppresses the Romo1 protein from binding to a complex II member selected from the group consisting of RIP1, TRAF2, TRADD, FADD and a combination thereof or from associating with Bcl-XL in higher efficiency compared to the control.

7. A method for diagnosing a TNF-α-induced disease, comprising determining an mRNA level of a gene having a nucleotide sequence of SEQ ID NO. 1 or an analog thereof.

8. The method according to claim 7, wherein the TNF-α-induced disease is selected from the group consisting of sepsis, diabetes, osteoporosis, allograft rejection, multiple sclerosis, rheumatoid arthritis, inflammatory bowel diseases, ischemia/reperfusion injury, pulmonary fibrosis, ischemic stroke, Alzheimer's disease, Parkinson's disease and a combination thereof.

9. The method according to claim 7, wherein the mRNA level is determined by quantitatively measuring an oligonucleotide which hybridizes at least partially with the gene.

10. A method for diagnosing a TNF-α-induced disease, comprising determining an expression level of a protein having an amino acid of SEQ ID NO. 2 or an analog thereof.

11. The method according to claim 10, wherein the TNF-α-induced disease is selected from the group consisting of sepsis, diabetes, osteoporosis, allograft rejection, multiple sclerosis, rheumatoid arthritis, inflammatory bowel diseases, ischemia/reperfusion injury, pulmonary fibrosis, ischemic stroke, Alzheimer's disease, Parkinson's disease and a combination thereof.

12. A kit for diagnosing a TNF-α-induced disease using the method of one of claims 7 to 11, said TNF-α-induced disease being selected from the group consisting of sepsis, diabetes, osteoporosis, allograft rejection, multiple sclerosis, rheumatoid arthritis, inflammatory bowel diseases, ischemia/reperfusion injury, pulmonary fibrosis, ischemic stroke, Alzheimer's disease, Parkinson's disease and a combination thereof.


SEQUENCE LISTING (2 sequences)

SEQ ID NO: 1 - DNA, 391 bp, Unknown
  REACTIVE OXYGEN SPECIES MODULATOR 1
gagcgccctccccgtcgttttccgtgagagacgtagagctgagcgacccagcccgcgagcgaggtgagatgccggtggccgtgggtccctacggacagtcccagccaagctgcttcgaccgtgtcaaaatgggcttcgtgatgggttgcgccgtgggcatggcggccggggcgctcttcggcaccttttcctgtctcaggatcggaatgcggggtcgagagctgatgggcggcattgggaaaaccatgatgcagagtggcggcacctttggcacattcatggccattgggatgggcatccgatgctaaccatggttgccaactacatctgtcccttcccatcaatcccagcccatgtactaataaaagaaagtctttgagtaaaaaaaaaa

SEQ ID NO: 2 - protein, 79 aa, Unknown
  PROTEIN of REACTIVE OXYGEN SPECIES MODULATOR 1
MPVAVGPYGQSQPSCFDRVKMGFVMGCAVGMAAGALFGTFSCLRIGMRGRELMGGIGKTMMQSGGTFGTFMAIGMGIRC